(19) United States
(12) Patent Application Publication
(10) Pub. No.: US 2004/0101549 A1
(43) Pub. Date: May 27, 2004

(54) Treatments

(76) Inventor: Paul David Abel, London (GB)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/302,801

(22) Filed: Nov. 22, 2002

(51) Int. Cl. A61K 39/395; A61K 31/58; A61K 31/56; A61K 9/70

(52) U.S. Cl. 424/449; 514/182; 514/176

(57) ABSTRACT

A method for the treatment of carcinoma of the prostate is provided. A compound having estrogenic activity is administered transdermally to a subject in need of such treatment.

The present invention relates to the treatment of cancer, and in particular to treatment of carcinoma of the prostate.

Carcinoma of the prostate (CaP) is the second leading cause of cancer mortality in men. In the USA, 198000 cases are detected annually, of which 40% have disease that is no longer organ-confined and therefore not amenable to a surgical cure (Greenlee et al, CA Cancer J Clin 2001; 51(1):15-36). Androgen suppression (using LHRH agonists, non-steroidal antiandrogens, orchiectomy or a combination of these) is the treatment of choice for these patients and achieves symptomatic palliation in approximately 70-80% of cases. However, this type of hormonal therapy is also associated with significant morbidity, including symptoms of the andropause (the equivalent of the female menopause) and osteoporosis. Originally, oral estrogens were used for androgen suppression, achieving tumour response rates better than orchiectomy and equivalent to maximal androgen blockade, but they were abandoned because of cardiovascular toxicity (Aro. Prostate 1991; 18(2):131-137; Hedlund & Henriksson, The Scandinavian Prostatic Cancer Group (SPCG)-5 Trial Study. Urology 2000; 55(3):328-333; van Kesteren et al, Clin Endocrinol (Oxf) 1997; 47(3):337-342). By contrast, recent reports suggest that intramuscular estrogens reduce cardiovascular mortality (Aro. Prostate 1991; 18(2):131-137; Hedlund & Henriksson, The Scandinavian Prostatic Cancer Group (SPCG)-5 Trial Study. Urology 2000; 55(3):328-333; Mikkola et al, Finnprostate Group. Br J Urol 1998; 82(1):63-68). Intramuscular polyestradiol phosphate (PEP) has been piloted as first line CaP therapy in studies from Scandinavia. PEP reduced the short-term cardiovascular morbidity by over 90% (Mikkola et al, Finnprostate Group. Br J Urol 1998; 82(1):63-68), and conferred significant long-term cardiovascular protection in an epidemiological comparison to an age-standardized population (Aro. Prostate 1991; 18(2):131-137). The overall and cardiovascular mortality was equivalent in 915 patients randomized to PEP or maximum androgen blockade over 6 years (Hedlund & Henriksson, The Scandinavian Prostatic Cancer Group (SPCG)-5 Trial Study. Urology 2000; 55(3):328-333). These data are consistent with that established for coronary risk using hormone replacement therapy (HRT) in postmenopausal women (Hulley et al, Heart and Estrogen/progestin Replacement Study (HERS) Research Group. JAMA 1998; 280(7):605-613). However, monthly intramuscular PEP depots are painful and cannot be removed if toxicity develops.

In one aspect, the present invention provides a method for the treatment of carcinoma of the prostate, comprising administering to a subject in need of such treatment a compound having estrogenic activity, wherein the compound is administered transdermally.

By “carcinoma of the prostate” is meant a malignant tumour of the epithelial lining of the lumina of the prostate.

The compound having estrogenic activity may be an estrogen. Such estrogens may be any of those conventionally available which can be absorbed through the skin. Typically, the estrogen may be selected from the group comprising synthetic and natural estrogens. The synthetic estrogens may be selected from, for example, ethinyl estradiol, micronized estradiol, 17-β estradiol, mestranol, estradiol valerate, 11-nitrato estradiol, 7-α-methyl-11-nitrato-estradiol, piperazine estrone sulfate, quinestranol, polyestradiol phosphate (PEP), and 17α-ethinylestradiol and esters and ethers thereof.

The present inventors have found that transdermal administration of compounds having estrogenic activity can suppress serum androgens and PSA levels, reduce cardiovascular risk, prevent osteoporosis and improve the quality of life (QOL) for men with advanced CaP. Such therapy overcomes the problems associated with the known intramuscular therapy. Intramuscular injections are painful and need to be given frequently (about at least every 2 weeks) deeply into muscle. This requires ancillary staff to administer the injections, as well as a needle and syringe, resulting in increased costs. By contrast, transdermal administration can simply be applied by the patient. Also, should estrogen toxicity occur, intramuscular deposits cannot be easily removed and can continue to give problems, whereas transdermal administration can simply be ceased with consequent serum wash out within about 24 hours. Intravenous administration of estrogens is impractical as it would need to be given as often as daily to maintain serum levels and would require expert clinical skills.

Furthermore, in conventional anti-CaP therapy (LHRH), levels of FSH recover after 3-6 months. By contrast, with transdermal administration, FSH remains suppressed throughout the treatment. This is important because FSH stimulation has been shown to cause cell proliferation in cellular and in vivo models of prostate cancer independently of androgen receptor. This suggests that there are probably FSH receptors or other receptors which bind FSH on (at least some) prostate cancer cells which, when bound, cause cell proliferation. Thus, suppressing FSH may provide an additional mechanism to prevent or reduce prostate cancer cell proliferation and give an additional survival advantage to the patient.

Transdermal administration may be by means of a patch. Such patches are well-known to those of skill in the art, and examples include: Dermestril™ (Sanofi); Estraderm™ (Novartis); Evorel™ (Janessen-Cilag); Fematrix™ (Solvay); Femseven™ (Merck); Menorest™ (Rhone-Poulenc). Each new patch may be applied to a different site. Preferred application sites are clean, dry and intact areas of skin, below the waistline on the trunk of the body. Preferably the buttock, hip or abdomen area is chosen since these areas of skin wrinkle least during body movement. Alternatively or additionally, transdermal administration of the compound having estrogenic activity may be by means of ointment, cream, suspension, lotion, powder, solution, paste, gel, spray, aerosol, oil or other composition rubbed on or otherwise applied to the skin. Again, these are known to those of skill in the art, and examples include Oestrogel™ (Hoechst) and Sandrena™ (Organon) which are rubbed into the skin.

The compound having estrogenic activity may be administered so as to reduce levels of testosterone in the subject to castrate levels (e.g. <3 nmol/l, <2 nmol/l of blood or lower), and then to maintain testosterone at these levels. Reduction of testosterone to castrate levels may be achieved by administering sufficient compound having estrogenic activity over a period of weeks, such as 5-10, 6-9, or 8 weeks. How much is sufficient can be determined by one of skill in the art by monitoring blood levels of testosterone. In one embodiment, six transdermal estradiol (7.8 mg) patches are applied weekly for 5-10, 6-9, or 8 weeks.

Maintenance of castrate levels may be achieved by maintaining blood levels of estradiol in the range of about 600-2000 pmol/l or higher, 800-1500 pmol/l, 900-1200 pmol/l, or around 1000 pmol/l. When the method of the invention involves the use of transdermal patches, such levels of estradiol may be maintained using 1, 2, 3, 4, 5, 6 or more patches per week, depending on the dose in the patch. The patches (which may be estradiol (7.8 mg) patches) may be changed twice weekly to boost trough estradiol levels, and maintain patch adhesion and patient compliance.

Transdermal administration of compounds having estrogenic activity may be used in conjunction with other treatments for CaP, such as radiotherapy. In addition, after sufficient time to establish that a patient shows no toxicity to estrogenic compounds (for example about 12 months), such compounds may be administered subcutaneously, sub-lingually, transbuccally and/or transrectally. Such administration may take the place of transdermal administration or may be used together with transdermal administration. An example of a suitable sub-cutaneous implant is that made by Organon. The advantage of this is that these other routes of administration are less expensive.

In further aspects, the present invention provides a method for the treatment of carcinoma of the prostate, comprising administering to a subject in need of such treatment a compound having estrogenic activity, wherein the compound is administered subcutaneously, sub-lingually, transbuccally and/or transrectally.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986). Other suitable pharmaceutical compositions may be formulated as ointments, creams, suspensions, lotions, powders, solutions, pastes, gels, sprays, aerosols or oils. When formulated in an ointment, the active ingredient may be employed with either a paraffinic or a water-miscible ointment base. Alternatively, the active ingredient may be formulated in a cream with an oil-in-water cream base or a water-in-oil base.

Preferred features of each aspect of the invention are as for each of the other aspects mutatis mutandis. The prior art documents mentioned herein are incorporated to the fullest extent permitted by law.

### EXAMPLE

One embodiment of the present invention will now be described with reference to the following non-limiting example. Reference is made to the accompanying drawings in which:

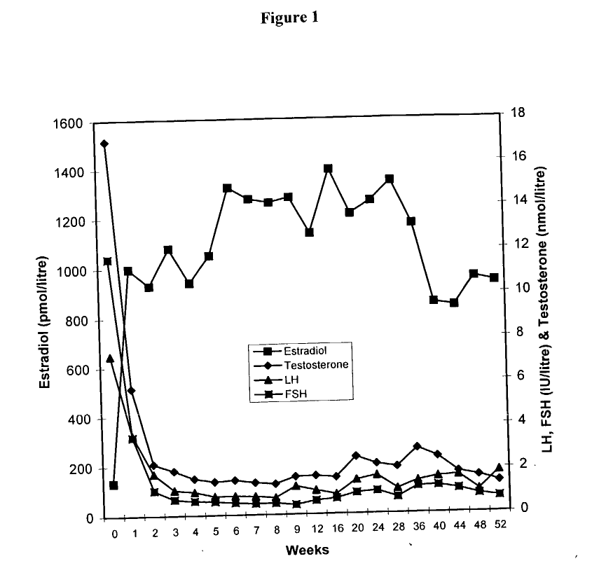

FIG. 1 is a graph showing the levels of hormones over time of transdermal estradiol therapy; and

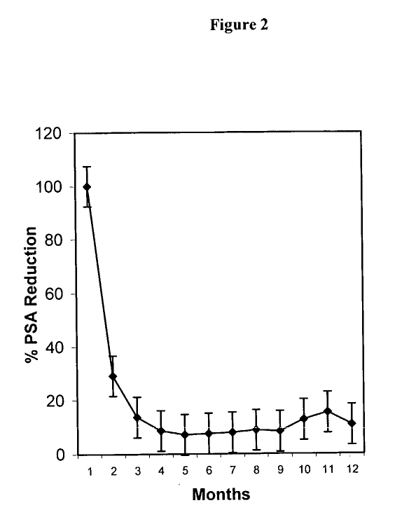

FIG. 2 is a graph showing the percentage PSA (mean and standard error) reduction against time, following transdermal estradiol therapy.

Patients, Methods and Results: Local ethics committees approved the study and all patients gave informed consent. Twenty patients with locally advanced (n=10) or metastatic (n=10) CaP were enrolled between August 2000 and March 2001. Six transdermal estradiol (7.8 mg) patches manufactured by Schering Healthcare were applied weekly for 8 weeks, and then reduced to maintain castrate levels of testosterone.

Hormone, PSA and thrombophilic assays were performed before treatment and at regular intervals thereafter. Vascular flow studies, CT and isotope bone scanning, and bone densitometry were performed 6 monthly to monitor for thrombosis, disease progression and osteoporosis respectively. All tests were carried out according to routine protocols known to those of skill in the art. All patients completed the European Organisation for Research and Treatment of Cancer (EORTC) QLQ-C30 cancer and PR25 prostate-specific QOL questionnaires.

Median follow up is 15 months (range 12 to 20 months). The patches were well tolerated by all patients. Estradiol levels greater than 1000 pmol/l were maintained using 2 patches (2 patches per week were sufficient to maintain castrate testosterone, but were changed twice weekly to boost trough estradiol levels, and maintain patch adhesion and patient compliance) and higher levels by increasing their number. All patients achieved castrate level of testosterone within 3 weeks (FIG. 1), and LH and FSH levels were suppressed to ensure that castrate levels were maintained, and had biochemical evidence of disease regression (see FIG. 2), with an average reduction in PSA of 95.1% (range 84.2-99.8%). One patient had biochemical relapse and died of his disease at 14 months. One patient had fluid retention and was withdrawn at 10 months, but no other cardiovascular toxicity occurred. None of the coagulation activators, inhibitors or fibrinolytic factors was induced by transdermal estradiol therapy, and elevated levels of prothrombin, fibrinogen and D-dimer at baseline were decreased (p<0.001; <0.001; 0.049 respectively). Arterial inflow was increased (p=0.004), whilst venous outflow was unaffected, and arterial compliance improved (p=0.049). Bone mineral densities of lumbar spine and hip were both significantly improved (p=0.05; 0.031), such that 4 patients had upgraded World Health Organization (WHO) classification of their osteoporotic risk. No patient had hot flushes. All other QOL parameters either stabilised or improved, except those relating to gynecomastia, which caused mild to moderate distress in 80%. Clinically significant improvements were noted in functional role and emotion domains as well as overall QOL, and these benefits were maintained throughout the study period.

Comment: This report demonstrates objective evidence of tumour regression and improved QOL for advanced CaP patients. Although oral estrogens were used historically for CaP, their use has been limited by the risk of severe cardiovascular and thromboembolic toxicity, in over 30% of patients. This toxicity occurs within the first year (the majority within 6 months) of initiating therapy (Aro. Prostate 1991; 18(2):131-137; Hedlund & Henriksson, The Scandinavian Prostatic Cancer Group (SPCG)-5 Trial Study. Urology 2000; 55(3):328-333). If these toxicity levels were extrapolated to this study, at least 6 or 7 cardiovascular complications would have been expected. This did not occur (Exact Clopper-Pearson confidence interval for this patient cohort is 0-17% i.e. at least 3 patients). In fact, only one episode (of fluid retention) was documented during the study period. It is now clear that the route of administration is crucial to the development of estrogen toxicity. Exposure of the liver to high doses of estrogen via the portal circulation alters the metabolism of hormones, coagulation proteins and lipids. The disruption to the physiological ratios of hormones, in particular, may be responsible for short and long-term cardiovascular effects. Activation of coagulation results in a significant increase in the risk of venous thrombosis. In contrast, parenteral estradiol delivery reduces the first pass exposure of the portal circulation to high concentrations of estrogen, and appears to minimise these risks (Aro. Prostate 1991; 18(2):131-137). In our study, the physiological ratios of estrone to estradiol, SHBG, LDL to HDL, factor VII and factor XII were maintained (data not shown), and elevated levels of prothrombin, fibrinogen and D-dimer were decreased. As a result, transdermal estradiol therapy reversed the hypercoagulable state often associated with advanced CaP, and induced by oral estrogens (Carlstrom et al, Prostate 1989; 14(2):177-182; von Schoultz et al, Prostate 1989; 14(4):389-395; Henriksson et al, Br J Urol 1990; 65(3):282-285). Moreover, transdermal estradiol therapy improved the vascular flow and increased arterial compliance, changes suggestive of a cardiovascular benefit (Hulley et al, Heart and Estrogen/progestin Replacement Study (HERS) Research Group. JAMA 1998; 280(7):605-613).

Transdermal delivery avoids of the problems of pain and difficulty of removal associated with intramuscular depots of estrogens. Transdermal estrogens have been used successfully in gender reassignment patients, specifically to negate thromboembolic complications (van Kesteren et al, Clin Endocrinol (Oxf) 1997; 47(3):337-342). High dose parenteral estrogen therapy may have a direct and independent toxicity on prostate cancer cells, potentially increasing time to relapse and/or delaying mortality (Ferro, Urol Clin North Am 1991; 18(1):139-143).

Conventional modalities of androgen suppression cause distressing symptoms of andropause, especially hot flushes in over 70% (Spetz et al, J Urol 2001; 166(2):517-520). Other effects of castration include anaemia, fatigue, depression, dementia and muscle wasting. Together, these side effects adversely affect the QOL for advanced CaP patients. Transdermal estrogens, in the form of HRT have been used to minimize similar symptoms in women and improve the QOL for menopause sufferers. Transdermal estrogens protect against cardiovascular disease, menopausal symptoms and osteoporosis in women (Mijatovic & Pines, Maturitas 1999; 33(1):87-92). Our data suggest that similar benefits can be conferred to men with CaP, contrary to the prevailing expectation that there would be severe cardiovascular side effects. Androgen suppression also induces accelerated osteoporosis. Bone losses varying from 3 to 10% per year have been reported, resulting in increased frequency of osteoporotic fracture (Daniell et al, J Urol 2000; 163(1):181-186). Regular intravenous biphosphonate therapy has been proposed to prevent bone loss (Smith et al, N Engl J Med 2001; 345(13):948-955). In this study, transdermal estradiol therapy increased the bone density sufficiently to improve the WHO classification of several at risk patients (data not shown). This will become an increasingly important benefit, especially as early androgen suppression is increasingly preferred over deferred therapy (Walsh et al J Urol 2001; 166(2):508-515).

Transdermal estrogens are an effective treatment for advanced CaP, producing castrate levels of testosterone and objective clinical response. They are acceptable to patients with minimal morbidity (gynecomastia), and protect against andropause, hot flushes and osteoporosis. A major proportion of the $1.5 billion expenditure for the treatment of CaP in the USA is used on hormonal interventions (Walsh et al, J Urol 2001; 166(2):508-515.) Transdermal estrogen therapy costs less than one-tenth of LHRH agonists, potentially saving over $900 million annually in the USA alone.

1. A method for the treatment of carcinoma of the prostate, comprising administering to a subject in need of such treatment a compound having estrogenic activity, wherein the compound is administered transdermally.

2. The method of claim 1, wherein the compound is an estrogen.

3. The method of claim 2, wherein the estrogen is selected from the group comprising synthetic and natural estrogens.

4. The method of claim 3, wherein the synthetic estrogen is selected from ethinyl estradiol, micronized estradiol, 17-β estradiol, mestranol, estradiol valerate, 11-nitrato estradiol, 7-α-methyl-11-nitrato-estradiol, piperazine estrone sulfate, quinestranol, polyestradiol phosphate (PEP), and 17α-ethinylestradiol and esters and ethers thereof.

5. The method of claim 2, wherein the estrogen is estradiol.

6. The method of claim 1, wherein the compound is administered by means of a patch.

7. The method of claim 1, wherein the compound is administered by means of a compound applied to the skin.

8. The method of claim 1, wherein the compound having estrogenic activity is administered so as to reduce levels of testosterone in the subject to castrate levels, and then to maintain testosterone at these levels.

9. The method of claim 1, wherein transdermal administration of said compound having estrogenic activity is replaced or supplemented with subcutaneous, sub-lingual, transbuccal and/or transrectal administration of said compound, provided said subject shows no toxicity to said compound.
